(19) United States
(12) Patent Application Publication
(10) Pub. No.: US 2010/0311776 A1
(43) Pub. Date: Dec. 9, 2010

(54) Novel sEH Inhibitors and their Use

(75) Inventors: Joseph Paul Marino, JR., King of Prussia, PA (US); John Jeffrey McAtee, King of Prussia, PA (US)

Correspondence Address: GlaxoSmithKline;GLOBAL PATENTS -US, UW2220, P. O. BOX 1539, KING OF PRUSSIA, PA, 19406-0939, US

(73) Assignee: Smithkline Beecham Corporation

(21) Appl. No.: 12/864,721

(22) PCT Filed: Jan. 30, 2009
(86) PCT No.: PCT/US09/32526

§ 371 Date: Jul. 27, 2010

Related U.S. Application Data
(60) Provisional application No. 61/024,609, filed on Jan. 30, 2008.

(51) Int. Cl. A61K 31/506; C07D 401/04; A61P 9/12; A61P 9/10; A61P 11/06; A61P 11/00; A61P 3/10; A61P 3/04

(52) U.S. Cl. 514/275; 544/329; 514/256

(57) ABSTRACT

The invention is directed to novel sEH inhibitors and their use in the treatment of diseases mediated by the sEH enzyme. Specifically, the invention is directed to compounds according to Formula I:

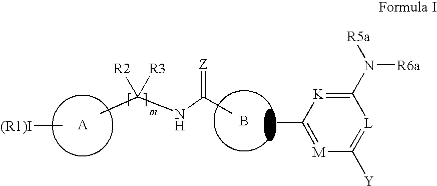

wherein R1, R2, R3, R5a, R6a, A, B, K, L, M, Y, Z, l, and m are defined herein, and to pharmaceutically-acceptable salts thereof. The compounds of the invention are sEH inhibitors and can be used in the treatment of diseases mediated by the sEH enzyme, such as hypertension. Accordingly, the invention is further directed to pharmaceutical compositions comprising a compound of the invention. The invention is still further directed to methods of inhibiting sEH and treatment of conditions associated therewith using a compound of the invention or a pharmaceutical composition comprising a compound of the invention.

### FIELD OF THE INVENTION

The invention is directed to novel sEH inhibitors and their use in the treatment of diseases mediated by the sEH enzyme.

### BACKGROUND OF THE INVENTION

Epoxide functional groups may be found in drugs, xenobiotic materials, and endogenous biomolecules. Epoxide hydrolases, found in both plants and animals, are enzymes that convert epoxides to diols by hydrolysis. In mammals, soluble epoxide hydrolase (“sEH”) is primarily responsible for the metabolism of arachidonic acid derivatives known as epoxyeicosatrienoic acids (“EETs”). sEH converts EETs into dihydroxyeicosatrienoic acids (“DHETs”). Several publications have described the beneficial vasodilatory, anti-inflamatory, and anti-thrombotic effects of EETs. Spector et al., Prog. Lipid Res., 43, 55-90, 2004; Imig, Cardiovasc. Drug Rev., 24, 169-188, 2006. DHETs are generally inactive and thus do not exhibit the beneficial effects of EETs.

Conversely, microsomal epoxide hydrolase (“mEH”) catalyzes the hydrolysis of a broad range of epoxide substrates including carcinogenic polycyclic aromatic hydrocarbons and reactive epoxides, thus it provides an important detoxification pathway. Polymorphisms in mEH may lead to differences in bioactivation of pro-carcinogens and several human epidemiological studies suggest that mEH genotype is associated with altered cancer risk. Fretland & Omiecinski, Chemico-Biol. Int., 129, 41-59, 2000.

Pharmacological, knockout mouse phenotype and genetic polymorphism studies suggest that elevated EET levels are protective in numerous disorders including hypertension [Cell Biochem Biophys., 47, 87-98, 2007], heart failure [Xu et al., Proc. Natl. Acad. Sci. U.S.A., 103, 18733-18738, 2006], renal dysfunction/end organ damage [Zhao et al. J. Am. Soc. Nephrol., 15, 1244-1253, 2004; Imig et al., Hypertension, 46, 975-981, 2005], stroke [Koerner et al., J. Neurosci., 27; 4642-4649, 2007], atherosclerosis and thrombosis [Wei et al., Atherosclerosis, 190, 26-34, 2007; Krotz et al., Arterioscler. Thromb. Vasc. Biol., 24; 595-600, 2004] and inflammation [Inceoglu et al., Life Sci., 79, 2311-2319, 2006]. One approach to the treatment of such conditions designed to take advantage of the beneficial effect of EETs has been to search for compounds that inhibit sEH thereby preventing EET degradation.

### SUMMARY OF THE INVENTION

The invention is directed to novel sEH inhibitors and their use in the treatment of diseases mediated by the sEH enzyme. Specifically, the invention is directed to compounds according to Formula I:

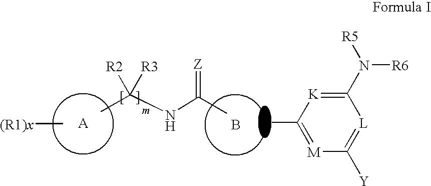

wherein R1, R2, R3, R5, R6, A, B, K, L, M, Y, Z, x, and m are defined below, and to pharmaceutically-acceptable salts thereof.

In yet another aspect, this invention provides for the use of the compounds of Formula (I) for the treatment or prevention of hypertension, organ failure/damage (including heart failure, renal failure, cardiac and renal fibrosis, and liver failure), peripheral vascular disease (including ischemic limb disease, intermittent claudication, endothelial dysfunction, erectile dysfunction, Raynaud's disease, and diabetic vasculopathies e.g. retinopathy), atherosclerosis, atherothrombotic disorders (including coronary artery disease, coronary vasospasm, angina, stroke, myocardial ischemia, myocardial infarction, and hyperlipidemia), metabolic disorders (including diabetes, metabolic syndrome, hyperglycemia, and obesity), inflammation, inflammatory disorders (including arthritis, inflammatory pain, overactive bladder, asthma, and COPD), cognitive disorders (including cognitive impairment, dementia, and depression), glaucoma, osteoporosis, and polycystic ovary syndrome.

The compounds of this invention may be administered alone or in conjunction with one or more other therapeutic agents, eg. agents being selected from the group consisting of may be administered alone or in conjunction with one or more other therapeutic agents, eg. agents being selected from the group consisting of endothelin receptor antagonists, angiotensin converting enzyme (ACE) inhibitors, angiotension II receptor antagonists, vasopeptidase inhibitors, diuretics, digoxin, beta blocker, aldosterone antagonists, iontropes, NSAIDS, nitric oxide donors, calcium channel modulators, muscarinic antagonists, steroidal anti-inflammatory drugs, bronchodilators, Leukotriene antagonist, HMG-CoA reductase inhibitors, dual non-selective β-adrenoceptor and α₁ adrenoceptor antagonists, type-5 phosphodiesterase inhibitors, and renin inhibitors.

### DETAILED DESCRIPTION OF THE INVENTION

### Compounds

The invention is directed to compounds according to Formula I:

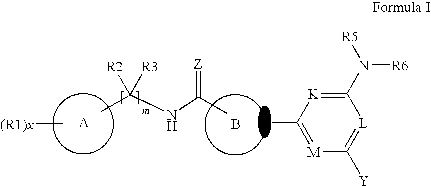

wherein:

A is phenyl, monocyclic heteroaryl, or C5-C6 cycloalkyl;

when A is phenyl or monocyclic heteroaryl each R1 is selected from the group consisting of: halo, —CN, R14, R15, R16, R17, R18, R19, —ORb, —C(O)ORc, —C(O)NRcRc, —NRcRc, —NRcC(O)Rb, —NRcS(O₂)Ra, —SRb, —S(O₂)Ra, and —S(O₂)NRcRc;

when A is C5-C6 cycloalkyl each R1 is selected from the group consisting of: Ra, —ORb, —C(O)ORc, —C(O)NRcRc, —NRcRc, and —NRcC(O)Rb;

each R14 is C1-C6 alkyl optionally substituted with one or more substituents selected from the group consisting of: halo, —ORd, and —NRfRf;

each R15 is C3-C6 cycloalkyl optionally substituted with one or more substituents selected from the group consisting of: halo, —ORd, —NRfRf, and C1-C3 alkyl;

each R16 is monocyclic heterocycloalkyl optionally substituted with one or more C1-C3 alkyl;

each R17 is phenyl optionally substituted with one or more substituents selected from the group consisting of: halo, —CN, C1-C3 alkyl, C1-C3 haloalkyl, —ORd, —NRfRf, and —S(O₂)Ra;

each R18 is monocyclic heteroaryl optionally substituted with one or more substituents selected from the group consisting of: halo, —CN, C1-C3 alkyl, C1-C3 haloalkyl, —ORd, —NRfRf, and —S(O₂)Ra;

each R19 is C1-C3 alkyl substituted with R15, R16, R17, or R18;

x is an integer from 0 to 5;

each R2 is H or C1-C3 alkyl;

each R3 is H or C1-C3 alkyl;

m is 1 or 2;

Z is O or S;

B is B1, B2, B3, B4, or B5 wherein

B1 is

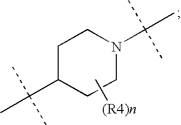

B2 is

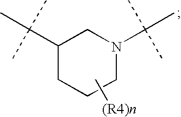

B3 is

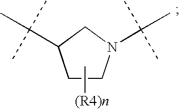

B4 is

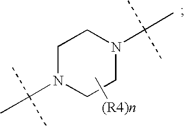

B5 is

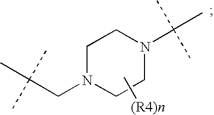

each R4 is C1-C3 alkyl;

n is an integer from 0 to 4;

either K and L are each N and M is CR13, or L and M are each N and K is CR13;

Y is C1-C8 alkyl optionally substituted with one or more substituents selected from the group consisting of: halo, —ORd, —SRd, —NReRe, C3-C6 cycloalkyl, Rh, Ri, and Rj;

R5 is H, R51, R52, R53, R54, R55, —C(O)Rb, —C(O)NRcRc, —S(O₂)Ra, or —S(O₂)NRcRc;

each R51 is C1-C6 alkyl optionally substituted with one or more substituents selected from the group consisting of: halo, —ORd, —SRk, —C(O)ORc, —C(O)NReRe, —NReRe, Rg, Rh, Ri, Rj;

R52 is C3-C6 cycloalkyl optionally substituted with one or more substituents selected from the group consisting of: halo, —ORd, —SRd, —C(O)ORc, —C(O)NReRe, —NReRe, C1-C3 alkyl, and C1-C3 haloalkyl;

R53 is monocyclic heterocycloalkyl optionally substituted with one or more C1-C3 alkyl;

R54 is phenyl optionally substituted with one or more substituents selected from the group consisting of: halo, CN, Ra, —ORb, —C(O)ORc, —C(O)NRcRc, —NRcRc, —NRcC(O)Rb, —NRcS(O₂)Ra, —SRb, —S(O₂)Ra, and —S(O₂)NReRe;

R55 is monocyclic heteroaryl optionally substituted with one or more substituents selected from the group consisting of: halo, —CN, C1-C3 alkyl, C1-C3 haloalkyl, —ORd, —NReRe, and —S(O₂)Ra;

R6 is H or R51; or

R5 and R6 taken together with the nitrogen atom to which they are attached form a saturated monocyclic ring having from 5 to 7 member atoms wherein said ring optionally contains one additional heteroatom as a member atom and wherein said ring is optionally substituted with one or more substituents selected from the group consisting of: C1-C3 alkyl, —ORd, and —NRfRf;

R13 is H, R7, R8, R9, R10, R11, —C(O)ORc, —CONRlRl, —NRlRl, —NRcC(O)Rm, —NRc(SO₂)Rm;

R7 is C1-C8 alkyl optionally substituted with one or more substituents selected from the group consisting of: halo, —ORd, —C(O)ORc, —SRd, —NReRe, C3-C6 cycloalkyl, R1, and Rj;

R8 is C3-C6 cycloalkyl optionally substituted with one or more substituents selected from the group consisting of: halo, —ORd, —C(O)ORc, —SRd, —NReRe, C1-C3 alkyl, and C1-C3 haloalkyl;

R9 monocyclic heterocycloalkyl optionally substituted with one or more C1-C3 alkyl;

R10 is phenyl optionally substituted with one or more substituents selected from the group consisting of: halo, CN, Ra, —ORb, —C(O)ORc, —C(O)NReRe, —NReRe, —NRcC(O)Rb, —NRcS(O₂)Ra, —SRb, —S(O₂)Ra, and —S(O₂)NRcRc

R11 is heteroaryl optionally substituted with one or more substituents selected from the group consisting of: halo, CN, Ra, —ORb, —C(O)ORc, —C(O)NReRe, —NReRe, —NRcC(O)Rb, —NRcS(O₂)Ra, —SRb, —S(O₂)Ra, and —S(O₂)NRcRc;

each Ra is C1-C6 alkyl or C1-C6 haloalkyl;

each Rb is H, C1-C6 alkyl or C1-C6 haloalkyl;

each Rc is H or C1-C6 alkyl;

each Rd is H, C1-C3 alkyl or C1-C3 haloalkyl;

each Re is H, C1-C3 alkyl, —CH₂—CF₃; or

both Re groups, independently in each instance, taken together with the nitrogen atom to which they are attached form a saturated monocyclic ring having from 5 to 7 member atoms wherein said ring optionally contains one additional heteroatom as a member atom and wherein said ring is optionally substituted with one or more substituents selected from the group consisting of: C1-C3 alkyl, ORd, and NRfRf;

each Rf is H or C1-C3 alkyl.

each Rg is C3-C6 cycloalkyl optionally substituted with one or more substituents selected from the group consisting of: halo, —ORd, —SRd, —C(O)ORc, —C(O)NReRe, —NReRe, and C1-C3 alkyl;

each Rh is monocyclic heterocycloalkyl optionally substituted with one or more C1-C3 alkyl;

each Ri is phenyl optionally substituted with one or more substituents selected from the group consisting of: halo, —CN, C1-C3 alkyl, C1-C3 haloalkyl, —ORd, —NReRe, and —S(O₂)Ra;

each Rj is monocyclic heteroaryl optionally substituted with one or more substituents selected from the group consisting of: halo, —CN, C1-C3 alkyl, C1-C3 haloalkyl, —ORd, —NReRe, and —S(O₂)Ra;

each Rk is H, C1-C3 alkyl, C1-C3 haloalkyl, or benzyl optionally substituted with one or more substituents selected from the group consisting of: halo, —CN, C1-C3 alkyl, C1-C3 haloalkyl, —ORd, and —NReRe;

each Rl is H, Rh, Ri, Rj, or Rn; or

both Rl groups, independently in each instance, taken together with the nitrogen atom to which they are attached form a saturated monocyclic ring having from 5 to 7 member atoms wherein said ring optionally contains one additional heteroatom as a member atom and wherein said ring is optionally substituted with one or more substituents selected from the group consisting of: C1-C3 alkyl, —ORd, and —NRfRf;

Rm is Rh, Ri, Rj, or Rn; and

each Rn is —CH₂—C1-C4 haloalkyl or C1-C6 alkyl optionally substituted with one or more substituents selected from the group consisting of: Rh, R1, and Rj;

or a pharmaceutically acceptable salt thereof.

The meaning of any functional group or substituent thereon at any one occurrence in Formula I, or any subformula thereof, is independent of its meaning, or any other functional group's or substituent's meaning, at any other occurrence, unless stated otherwise.

The compounds according to Formula I may contain one or more asymmetric centers (also referred to as a chiral center) and may, therefore, exist as individual enantiomers, diastereomers, or other stereoisomeric forms, or as mixtures thereof. Chiral centers, such as chiral carbon atoms, may also be present in a substituent such as an alkyl group. Where the stereochemistry of a chiral center present in Formula I, or in any chemical structure illustrated herein, is not specified the structure is intended to encompass any stereoisomer and all mixtures thereof. Thus, compounds according to Formula I containing one or more chiral center may be used as racemic mixtures, enantiomerically enriched mixtures, or as enantiomerically pure individual stereoisomers.

Individual stereoisomers of a compound according to Formula I which contain one or more asymmetric center may be resolved by methods known to those skilled in the art. For example, such resolution may be carried out (1) by formation of diastereoisomeric salts, complexes or other derivatives; (2) by selective reaction with a stereoisomer-specific reagent, for example by enzamatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation.

The compounds according to Formula I may also contain double bonds or other centers of geometric asymmetry. Where the stereochemistry of a center of geometric asymmetry present in Formula I, or in any chemical structure illustrated herein, is not specified, the structure is intended to encompass the trans (E) geometric isomer, the cis (Z) geometric isomer, and all mixtures thereof. Likewise, all tautomeric forms are also included in Formula I whether such tautomers exist in equilibrium or predominately in one form.

In certain embodiments, compounds according to Formula (I) may contain a basic functional group and are therefore capable of forming pharmaceutically acceptable acid addition salts by treatment with a suitable acid. Suitable acids include pharmaceutically acceptable inorganic acids and organic acids. Representative pharmaceutically acceptable acids include hydrogen chloride, hydrogen bromide, nitric acid, sulfuric acid, sulfonic acid, phosphoric acid, acetic acid, hydroxyacetic acid, phenylacetic acid, propionic acid, butyric acid, valeric acid, maleic acid, acrylic acid, fumaric acid, malic acid, malonic acid, tartaric acid, citric acid, salicylic acid, benzoic acid, tannic acid, formic acid, stearic acid, lactic acid, ascorbic acid, p-toluenesulfonic acid, oleic acid, lauric acid, and the like.

In certain embodiments, compounds according to Formula I may contain an acidic functional group and are therefore capable of forming pharmaceutically-acceptable base addition salts by treatment with a suitable base. Thus, the skilled artisan will appreciate that pharmaceutically-acceptable salts of the compounds according to Formula I may be prepared. Indeed, in certain embodiments of the invention, pharmaceutically-acceptable salts of the compounds according to Formula I may be preferred over the respective free base or free acid because such salts impart greater stability or solubility to the molecule thereby facilitating formulation into a dosage form. Accordingly, the invention is further directed to pharmaceutically-acceptable salts of the compounds according to Formula I.

As used herein, the term “pharmaceutically-acceptable salts” refers to salts that retain the desired biological activity of the subject compound and exhibit minimal undesired toxicological effects. These pharmaceutically-acceptable salts may be prepared in situ during the final isolation and purification of the compound, or by separately reacting the purified compound in its free acid or free base form with a suitable base or acid, respectively.

As used herein, the term “compounds of the invention” means both the compounds according to Formula I and the pharmaceutically-acceptable salts thereof. The term “a compound of the invention” also appears herein and refers to both a compound according to Formula I and its pharmaceutically-acceptable salts.

In the solid state, compounds of the invention can exist in crystalline, semi-crystalline and amorphous forms, as well as mixtures thereof. The skilled artisan will appreciate that pharmaceutically-acceptable solvates of a compound of the invention may be formed wherein solvent molecules are incorporated into the solid-state structure during crystallization. Solvates may involve water or nonaqueous solvents, or mixtures thereof. In addition, the solvent content of such solvates can vary in response to environment and upon storage. For example, water may displace another solvent over time depending on relative humidity and temperature.

Solvates wherein water is the solvent that is incorporated into the solid-state structure are typically referred to as “hydrates.” Solvates wherein more than one solvent is incorporated into the solid-state structure are typically referred to as “mixed solvates”. Solvates include “stoichiometric solvates” as well as compositions containing variable amounts of solvent (referred to as “non-stoichiometric solvates”). Stoichiometric solvates wherein water is the solvent that is incorporated into the solid-state structure are typically referred to as “stoichiometric hydrates”, and non-stoichiometric solvates wherein water is the solvent that is incorporated into the solid-state structure are typically referred to as “non-stoichiometric hydrates”. The invention includes both stoichiometric and non-stoichiometric solvates.

In addition, crystalline forms of a compound of the invention, including solvates thereof, may contain solvent molecules, which are not incorporated into the solid-state structure. For example, solvent molecules may become trapped in the crystals upon isolation. In addition, solvent molecules may be retained on the surface of the crystals. The invention includes such forms.

The skilled artisan will further appreciate that compounds of the invention, including solvates thereof, may exhibit polymorphism (i.e. the capacity to occur in different crystalline packing arrangements). These different crystalline forms are typically known as “polymorphs.” The invention includes all such polymorphs. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different IR spectra and X-ray powder diffraction patterns, which may be used for identification. Polymorphs may also exhibit different melting points, which may be used for identification. The skilled artisan will appreciate that different polymorphs may be produced, for example, by changing or adjusting the reaction conditions or reagents, used in making the compound. For example, changes in temperature, pressure, or solvent may result in the production of different polymorphs. In addition, one polymorph may spontaneously convert to another polymorph under certain conditions.

### TERMS AND DEFINITIONS

“Alkyl” refers to a monovalent saturated hydrocarbon chain having the specified number of member atoms. For example, C1-C8 alkyl refers to an alkyl group having from 1 to 8 member atoms. Alkyl groups may be optionally substituted with one or more substituents as defined herein. Alkyl groups may be straight or branched. Representative branched alkyl groups have one, two, or three branches. Alkyl includes methyl, ethyl, propyl (n-propyl and isopropyl), butyl (n-butyl, isobutyl, and t-butyl), pentyl (n-pentyl, isopentyl, and neopentyl), and hexyl.

“Cycloalkyl” refers to a monovalent saturated or unsaturated hydrocarbon ring having the specified number of member atoms. For example, C3-C6 cycloalkyl refers to a cycloalkyl group having from 3 to 6 member atoms. Unsaturated Cycloalkyl groups have one or more carbon-carbon double bonds within the ring. Cycloalkyl groups are not aromatic. Cycloalkyl groups having from 3 to 7 member atoms or less are monocyclic ring systems. Cycloalkyl groups having at least 7 member atoms may be monocyclic, bridged or fused bicyclic ring systems. Cycloalkyl groups may be optionally substituted with one or more substituents as defined herein. Cycloalkyl includes cyclopropyl, cyclopropenyl, cyclobutyl, cyclobutenyl, cyclopentyl, cyclopentenyl, cyclohexyl, cyclohexenyl, cycloheptanyl, and cycloheptenyl.

“Enantiomerically enriched” refers to products whose enantiomeric excess is greater than zero. For example, enantiomerically enriched refers to products whose enantiomeric excess is greater than 50% ee, greater than 75% ee, and greater than 90% ee.

“Enantiomeric excess” or “ee” is the excess of one enantiomer over the other expressed as a percentage. As a result, since both enantiomers are present in equal amounts in a racemic mixture, the enantiomeric excess is zero (0% ee). However, if one enantiomer was enriched such that it constitutes 95% of the product, then the enantiomeric excess would be 90% ee (the amount of the enriched enantiomer, 95%, minus the amount of the other enantiomer, 5%).

“Enantiomerically pure” refers to products whose enantiomeric excess is 99% ee or greater.

“Half-life” refers to the time required for half of a quantity of a substance to be converted to another chemically distinct specie in vitro or in vivo.

“Halo” refers to the halogen radical fluoro, chloro, bromo, or iodo.

“Haloalkyl” refers to an alkyl group that is substituted with one or more halo substituents. Haloalkyl includes trifluoromethyl.

“Heteroaryl” refers to a monovalent aromatic ring containing from 1 to 4 heteroatoms as member atoms in the ring. Heteroaryl groups containing more than one heteroatom may contain different heteroatoms. Heteroaryl groups may be optionally substituted with one or more substituents as defined herein. Unless otherwise specified, heteroaryl groups are monocyclic ring systems or are fused, spiro, or bridged bicyclic ring systems. Monocyclic heteroaryl rings have 5 or 6 member atoms. Bicyclic heteroaryl rings have from 7 to 11 member atoms. Bicyclic heteroaryl rings include those rings wherein phenyl and a monocyclic heterocycloalkyl ring are attached forming a fused, spiro, or bridged bicyclic ring system, and those rings wherein a monocyclic heteroaryl ring and a monocyclic cycloalkyl, cycloalkenyl, heterocycloalkyl, or heteroaryl ring are attached forming a fused, spiro, or bridged bicyclic ring system. Heteroaryl includes pyrrolyl, pyrazolyl, imidazolyl, oxazolyl, isoxazolyl, oxadiazolyl, thiazolyl, isothiazolyl, thiadiazolyl, furanyl, furazanyl, thienyl, triazolyl, pyridinyl, pyrimidinyl, pyridazinyl, pyrazinyl, triazinyl, tetrazinyl, tetrazolyl, indolyl, isoindolyl, indolizinyl, indazolyl, purinyl, quinolinyl, isoquinolinyl, quinoxalinyl, quinazolinyl, pteridinyl, cinnolinyl, benzimidazolyl, benzopyranyl, benzoxazolyl, benzisoxazolyl, benzofuranyl, isobenzofuranyl, benzothiazolyl, benzisothiazolyl, benzothienyl, furopyridinyl, and napthyridinyl.

“Heteroatom” refers to a nitrogen, sulphur, or oxygen atom.

“Heterocycloalkyl” refers to a saturated or unsaturated ring containing from 1 to 4 heteroatoms as member atoms in the ring. However, heterocycloalkyl rings are not aromatic. Heterocycloalkyl groups containing more than one heteroatom may contain different heteroatoms. Heterocycloalkyl groups may be optionally substituted with one or more substituent as defined herein. Unless otherwise specified, heterocycloalkyl groups are monocyclic, bridged, or fused ring systems. Monocyclic heterocycloalkyl rings have from 4 to 7 member atoms. Bridged or bicyclic heterocycloalkyl rings have from 7 to 11 member atoms. In certain embodiments, heterocycloalkyl is saturated. In other embodiments, heterocycloalkyl is unsaturated but not aromatic. Heterocycloalkyl includes pyrrolidinyl, tetrahydrofuranyl, dihydrofuranyl, pyranyl, tetrahydropyranyl, dihydropyranyl, tetrahydrothienyl, pyrazolidinyl, oxazolidinyl, thiazolidinyl, piperidinyl, homopiperidinyl, piperazinyl, morpholinyl, thiamorpholinyl, azepinyl, 1,3-dioxolanyl, 1,3-dioxanyl, 1,4-dioxanyl, 1,3-oxathiolanyl, 1,3-oxathianyl, 1,3-dithianyl, azetidinyl, azabicylo[3.2.1]octyl, azabicylo[3.3.1]nonyl, azabicylo[4.3.0]nonyl, oxabicylo[2.2.1]heptyl, and pthalimidyl.

“Member atoms” refers to the atom or atoms that form a chain or ring. Where more than one member atom is present in a chain and within a ring, each member atom is covalently bound to an adjacent member atom in the chain or ring. Atoms that make up a substituent group on a chain or ring are not member atoms in the chain or ring.

“Optionally substituted” indicates that a group, such as alkyl, alkenyl, alkynyl, aryl, cycloalkyl, cycloalkenyl, heterocycloalkyl, or heteroaryl, may be unsubstituted or substituted with one or more substituents as defined herein. “Substituted” in reference to a group indicates that a hydrogen atom attached to a member atom within a group is replaced. It should be understood that the term “substituted” includes the implicit provision that such substitution be in accordance with the permitted valence of the substituted atom and the substituent and that the substitution results in a stable compound (i.e. one that does not spontaneously undergo transformation such as by rearrangement, cyclization, or elimination). A single atom may be substituted with more than one substituent as long as such substitution is in accordance with the permitted valence of the atom. Suitable substituents are defined herein for each substituted or optionally substituted group.

“Pharmaceutically acceptable” refers to those compounds, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

### Representative Embodiments

In one embodiment:

A is phenyl, thiophenyl, or pyridyl;

R1 is CF₃, halo, OCF₃, CN, OC₁-C₆ alkyl, morpholino, CO₂H, or N(CH₃)₂;

x is 1, 2, or 3;

B is B1, B2 or B3;

n is 0;

Z is O;

Y is C1-C3 alkyl;

R5 is hydrogen or C1-C6 alkyl;

R6 is hydrogen or C1-C6 alkyl;

K & L are N and M is CR13, or L & M are N and K is CR13;

R13 is hydrogen;

or a pharmaceutically acceptable salt thereof.

In another embodiment:

A is phenyl;

R1 is CF₃, halo, OCF₃, CN, OC₁-C₆ alkyl, or morpholino;

x is 1, or 2;

B is B1;

n is 0

Z is O;

Y is methyl;

R5 is hydrogen;

R6 is methyl;

K & L are N and M is CR13, or L & M are N and K is CR13;

R13 is hydrogen;

or a pharmaceutically acceptable salt thereof.

It is to be understood that the present invention covers all combinations of particular groups described hereinabove.

Specific examples of compounds of the present invention include the following:
N-[(2,4-dichlorophenyl)methyl]-1-[2-methyl-6-(methylamino)-4-pyrimidinyl]-4-piperidinecarboxamide;
1-[2-methyl-6-(methylamino)-4-pyrimidinyl]-N-{[2-(trifluoromethyl)phenyl]methyl}-4-piperidinecarboxamide;
1-[6-methyl-2-(methylamino)-4-pyrimidinyl]-N-{[2-(trifluoromethyl)phenyl]methyl}-4-piperidinecarboxamide; and
N-[(2,4-dichlorophenyl)methyl]-1-[6-methyl-2-(methylamino)-4-pyrimidinyl]-4-piperidinecarboxamide;
   or a pharmaceutically acceptable salt thereof.

### Compound Preparation

The compounds according to Formula I can be prepared using conventional organic syntheses. Suitable synthetic routes are depicted below in the following general reaction schemes. All functional groups are as defined in Formula I unless otherwise defined. Starting materials and reagents depicted below in the general reaction schemes are commercially available or can be made from commercially available starting materials using methods known by those skilled in the art.

The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups and methods for protecting and de-protecting different substituents using such suitable protecting groups are well known to those skilled in the art; examples of which may be found in T. Greene and P. Wuts, Protecting Groups in Chemical Synthesis (3rd ed.), John Wiley & Sons, NY (1999). In some instances, a substituent may be specifically selected to be reactive under the reaction conditions used. Under these circumstances, the reaction conditions convert the selected substituent into another substituent that is either useful as an intermediate compound or is a desired substituent in a target compound.

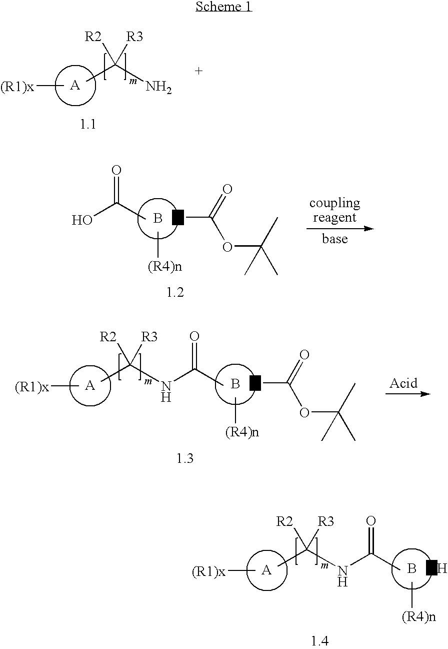

Scheme 1 represents a general reaction scheme for preparing intermediate 1.4. Treatment of compound 1.1 (commercially available or made from commercially available starting materials using methods known to those skilled in the art) with compound 1.2 (commercially available or made from commercially available starting materials using methods known to those skilled in the art) and a coupling reagent (such as BOP) and a base (such as triethylamine) in a solvent (such as DMF) provides intermediate 1.3. Treatment of compound 1.3 with an acid (such as trifluoroacetic acid) in a solvent (such as dichloromethane) provides intermediate 1.4.

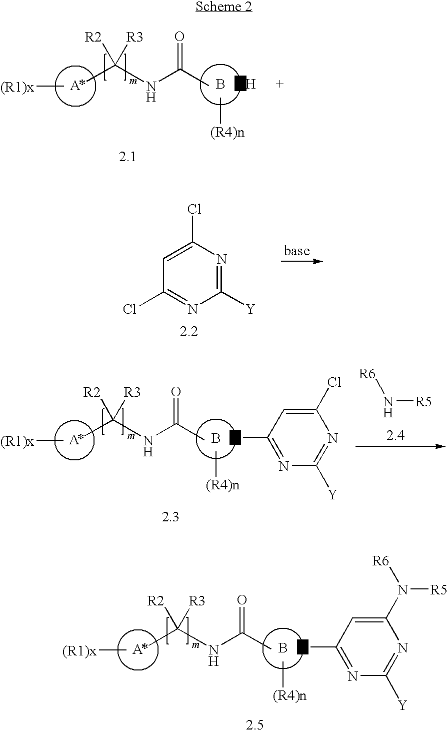

Scheme 2 represents a general reaction scheme for preparing certain compounds according to Formula I. Treatment of intermediate 2.1 (depicted above as intermediate 1.4) with compound 2.2 (commercially available or made from commercially available starting materials using methods known to those skilled in the art) with a base (such as aq NaOH) in a solvent (such as dioxane) at temperatures between 80° C. to 200° C. provides intermediate 2.3. Treatment of intermediate 2.3 with compound 2.4 (commercially available or made from commercially available starting materials using methods known to those skilled in the art) in a solvent (such as ethanol) at temperatures between 25° C. to 100° C. provides compounds according to Formula I wherein L and M are N and R13 is H, and Z is O (depicted as compound 2.5). Compound 2.5 can be converted to compounds according to Formula I wherein Z is S by methods known to those skilled in the art, such as by treatment with a thiolating agent (such as Lawesson's Reagent).

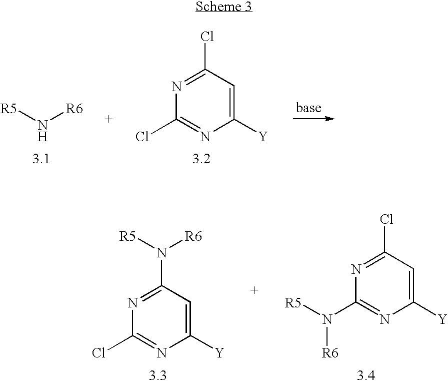

Scheme 3 represents a general reaction scheme for preparing intermediates 3.3 and 3.4. Treatment of compound 3.1 (commercially available or made from commercially available starting materials using methods known to those skilled in the art) with compound 3.2 (commercially available or made from commercially available starting materials using methods known to those skilled in the art) and a base (such as triethylamine) in a solvent (such as THF) at temperatures between −10° C. to 30° C. provides intermediates 3.3 and 3.4. Such isomers can be isolated by methods known to those skilled in the art.

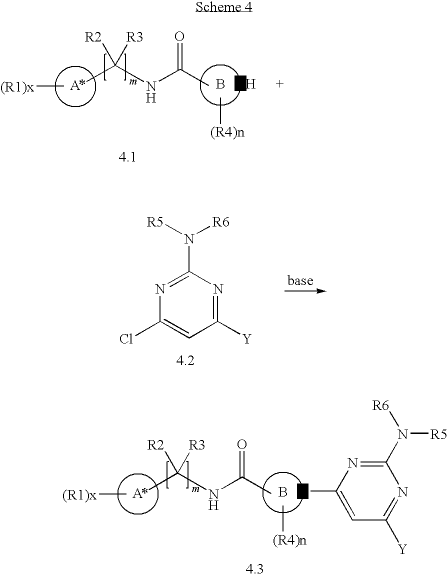

Scheme 4 represents a general reaction scheme for preparing certain compounds according to Formula I. Treatment of intermediate 4.1 (depicted above as intermediate 1.4) with compound 4.2 (depicted above as intermediate 3.4) with a base (such as aq NaOH) in a solvent (such as dioxane) at temperatures between 80° C. to 200° C. provides compounds according to Formula I wherein K and L are N and R13 is H, and Z is O (depicted as compound 4.3). Compound 4.3 can be converted to compounds according to Formula I wherein Z is S by methods known to those skilled in the art, such as by treatment with a thiolating agent (such as Lawesson's Reagent).

### EXAMPLES

The following examples illustrate the invention. These examples are not intended to limit the scope of the present invention, but rather to provide guidance to the skilled artisan to prepare and use the compounds, compositions, and methods of the present invention. While particular embodiments of the present invention are described, the skilled artisan will appreciate that various changes and modifications can be made without departing from the spirit and scope of the invention.

1H NMR spectra were recorded on a Bruker Avance 400 megahertz NMR spectrometer. Chemical shifts are expressed in parts per million (ppm, units). Coupling constants (J) are in units of hertz (Hz). Splitting patterns describe apparent multiplicities and are designated as s (singlet), d (doublet), t (triplet), q (quartet), dd (double doublet), dt (double triplet), m (multiplet), br (broad).

MS and liquid chromatography MS were recorded on a MDS Sciex liquid chromatography/mass spectroscopy system. All mass spectra were performed under electrospray ionization (ESI), chemical ionization (CI), electron impact (EI) or by fast atom bombardment (FAB) methods.

HPLC data was recorded on an Agilent 1100 series HPLC system with C-18 reverse phase column (Eclipse XDB-C18, 4.6×250 mm, 5 micron) running a gradient of 1-99% MeCN/H2O (+0.1% TFA) over 12 minutes.

All reactions were monitored by thin-layer chromatography on 0.25 mm E. Merck silica gel plates (60E-254), visualized with UV light, 5% ethanolic phosphomolybdic acid, p-anisaldehyde solution, aqueous potassium permanganate or potassium iodide/platinum chloride solution in water.

Flash column chromatography was performed on silica gel.

The naming program used is ACD Name Pro 6.02.

In describing the invention, chemical elements are identified in accordance with the Periodic Table of the Elements. Abbreviations and symbols utilized herein are in accordance with the common usage of such abbreviations and symbols by those skilled in the chemical and biological arts. For example, the following abbreviations are used herein:
“aq” is an abbreviation for aqueous
“BOC” is an abbreviation for tert-butoxycarbonyl
“BOP” is an abbreviation for (Benzotriazol-1-yloxy)tris (dimethylamino)phosphonium hexafluorophosphate
“° C.” is an abbreviation tion for degrees Celsius
“DMAP” is an abbreviation for dimethylaminopyridine
“DMF” is an abbreviation for dimethylformamide
“DMSO” is an abbreviation for Dimethylsulfoxide
“EDCI” is an abbreviation for N-(3-Dimethylaminopropyl)-N′-ethylcarbodiimide hydrochloride
“equiv” is an abbreviation for equivalent
“HPLC” is an abbreviation for High Pressure Liquid Chromatography
“g” is an abbreviation for gram or grams
“L” is an abbreviation for liter or liters
“LC-MS” is an abbreviation for Liquid chromatography-Mass spectrometry
“mL” is an abbreviation for milliliter or milliliters
“min” is an abbreviation for minute or minutes
“mmol” is an abbreviation for millimole or millimolar
“N” is an abbreviation for Normal and refers to the number of equivalents of reagent per liter of solution
“Ph” is an abbreviation for phenyl
“sat” is an abbreviation for saturated
“TFA” is an abbreviation for trifluoroacetic acid
“THF” is an abbreviation for tetrahydrofuran

### Intermediate 1

### N-{[2-(trifluoromethyl)phenyl]methyl}-4-piperidinecarboxamide

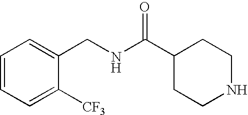

### Step 1: 1,1-dimethylethyl-4-[({[2-(trifluoromethyl)phenyl]methyl}amino)carbonyl]-1-Piperidinecarboxylate

A 500 mL round-bottom flask charged with argon was equipped with a magnetic stir bar, prior to the addition of 1-{[(1,1-dimethylethyl)oxy]carbonyl}-4-piperidinecarboxylic acid (14.31 g, 62.4 mmol), 2-(trifluoromethyl)benzylamine (8.79 mL, 62.4 mmol) and 100 mL of DMF at room temperature. Then, triethylamine (26.0 mL, 187.2 mmol) was added and the solution was allowed to stir for several minutes before a separate solution of 1H-1,2,3-benzotriazol-1-yloxy-tris(dimethylamino)-phosphonium hexafluorophosphate (BOP reagent, 27.6 g, 62.4 mmol) dissolved in 56 mL of DMF was delivered to the mixture at room temperature. The reaction was maintained at that temperature for 18 hours, before it was determined to be complete by LC-MS (m/e 387 [M+1]⁺). Pouring the crude mixture into a vigorously stirring 50/50 solution of saturated sodium bicarbonate and water (1.5 L), resulted in the precipitation of the desired product as an off-white solid. The solid was recovered by vacuum filtration and dried for 24 hours under vacuum to give 23.44 g of 1,1-dimethylethyl-4-[({[2-(trifluoromethyl)phenyl]methyl}amino)carbonyl]-1-piperidinecarboxylate (60.7 mmol, 97%). MS (ES) m/e 387 [M+H]⁺.

### Step 2: N-{[2-(trifluoromethyl)phenyl]methyl}-4-piperidinecarboxamide

A 500 mL round bottom flask equipped with a magnetic stir bar was charged with 1,1-dimethylethyl-4-[({[2-(trifluoromethyl)phenyl]methyl}amino)carbonyl]-1-piperidinecarboxylate (23.44 g, 60.7 mmol) and DCM (100 mL) at room temperature. Trifluoroacetic acid (100 mL) was added slowly, and the reaction was maintained at room temperature for 1 hour, before it was determined to be complete by LC-MS (m/e 288 [M+1]⁺). The volatiles were removed by rotary evaporation and the crude oil was dissolved in ethyl acetate and washed with saturated sodium bicarbonate solution (3×200 mL). The organic phase was dried over Na₂SO₄, filtered and concentrated to give 16.5 g of the title compound (57.7 mmol, 95%) as an off-white solid. MS (ES) m/e 288 [M+H]⁺.

### Intermediate 2

### N-[(2,4-dichlorophenyl)methyl]-4-piperidinecarboxamide

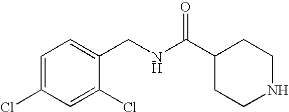

### Step 1: 1,1-dimethylethyl-4-({[(2,4-dichlorophenyl)methyl]amino}carbonyl)-1-piperidinecarboxylate

A 1000 mL round-bottom flask charged with argon was equipped with a magnetic stir bar, prior to the addition of 1-{[(1,1-dimethylethyl)oxy]carbonyl}-4-piperidinecarboxylic acid (16.32 g, 71.2 mmol), 2,4-dichlorobenzylamine (9.5 mL, 71.2 mmol) and 100 mL of DMF at room temperature. Afterwards, triethylamine (29.8 mL, 213.5 mmol) was added and the solution was allowed to stir for several minutes before a separate solution of 1H-1,2,3-benzotriazol-1-yloxy-tris(dimethylamino)-phosphonium hexafluorophosphate (BOP reagent, 31.5 g, 71.2 mmol) dissolved in 78 mL of DMF was delivered to the mixture at room temperature. The reaction was maintained at that temperature for 48 hours, before it was determined to be complete by LC-MS (m/e 388 [M+1]⁺). Pouring the crude mixture into a vigorously stirring 50/50 solution of saturated sodium bicarbonate and water (1.5 L), resulted in the precipitation of the desired product as an off-white solid. The solid was recovered by vacuum filtration and dried for 24 hours under vacuum to give 27.2 g of 1,1-dimethylethyl-4-({[(2,4-dichlorophenyl)methyl]amino}carbonyl)-1-piperidinecarboxylate (70.2 mmol, 98.6%). MS (ES) m/e 388 [M+H]⁺.

### Step 2: N-[(2,4-dichlorophenyl)methyl]-4-piperidinecarboxamide

A 500 mL round bottom flask equipped with a magnetic stir bar was charged with 1,1-dimethylethyl-4-({[(2,4-dichlorophenyl)methyl]amino}carbonyl)-1-piperidinecarboxylate (27.6 g, 71.2 mmol) and DCM (117 mL) at room temperature. Trifluoroacetic acid (117 mL) was added slowly, and the reaction was maintained at room temperature for 1 hour after which time LC-MS determined that the reaction was complete (m/e 287 [M+1]⁺). The volatiles were removed by rotary evaporation and the crude oil was dissolved in ethyl acetate and washed with saturated sodium bicarbonate solution (3×200 mL). The organic phase was dried over Na₂SO₄, filtered and concentrated to give 13.5 g of the title compound (47 mmol, 66%) as a pale yellow solid. MS (ES) m/e 287 [M+H]⁺.

### Example 1

### N-[(2,4-dichlorophenyl)methyl]-1-[2-methyl-6-(methylamino)-4-pyrimidinyl]-4-piperidinecarboxamide

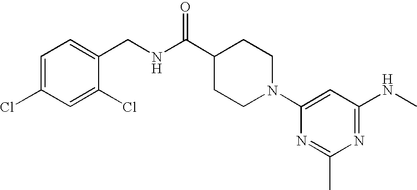

### Step 1: 1-(6-chloro-2-methyl-4-pyrimidinyl)-N-[(2,4-dichlorophenyl)methyl]-4-Piperidinecarboxamide

4,6-dichloro-2-methylpyrimidine (0.1 g, 0.613 mmol) and N-[(2,4-dichlorophenyl)methyl]-4-piperidinecarboxamide (0.176 g, 0.613 mmol) were combined in a 5.0 mL glass reaction tube that was equipped with a magnetic stir bar. The contents of the tube were taken up in 1,4-Dioxane (2 ml), and 1N sodium hydroxide (0.613 ml, 0.613 mmol) was added to the room temperature mixture and the contents were allowed to stir vigorously for 60 seconds. The tube was fitted with a rubber septum and hermetically sealed with a crimped metal foil seal. Using a Personal Chemistry Emrys Optimizer microwave unit, the reaction mixture was magnetically stirred and irradiated with microwave energy of dynamically adjusted power in order to maintain a temperature of 180° C. for one hour. Once the tube cooled to room temperature the reaction was determined to be complete by LC-MS (m/e 414 [M+1]⁺). Upon standing overnight a small amount of a pale yellow solid began to precipitate out of solution. The mixture was diluted to approximately 6 times the original reaction volume with water and more of the solid precipitated. The solid was collected by vacuum filtration and dried under vacuum overnight to give 84.8 mg of 1-(6-chloro-2-methyl-4-pyrimidinyl)-N-[(2,4-dichlorophenyl)methyl]-4-piperidinecarboxamide, (0.205 mmol, 33% yield) as a pale yellow solid. MS (ES) m/e 414 [M+H]+.

### Step 2: N-[(2,4-dichlorophenyl)methyl]-1-[2-methyl-6-(methylamino)-4-pyrimidinyl]-4-Piperidinecarboxamide

1-(6-chloro-2-methyl-4-pyrimidinyl)-N-[(2,4-dichlorophenyl)methyl]-4-piperidine carboxamide (0.0848 g, 0.205 mmol) and 33 wt. % methylamine in ethanol (1.531 ml, 12.30 mmol) were combined in a 5.0 mL glass reaction tube that was equipped with a magnetic stir bar. The tube was fitted with a rubber septum and hermetically sealed with a crimped metal foil seal. The mixture was stirred at 70° C. for 48 hours. Once the tube cooled to room temperature the reaction was determined to be complete by LC-MS (m/e 408 [M+1]⁺). Upon standing at room temperature the product precipitated as an off-white solid. The product was isolated by vacuum filtration and the solid was washed with cold ethanol. After drying in a vacuum oven overnight, 38.3 mg of the title compound (0.094 mmol, 46% yield) was recovered as an off-white solid. MS (ES) m/e 408 [M+H]⁺.

### Example 2

### 1-[2-methyl-6-(methylamino)-4-pyrimidinyl]-N-{[2-(trifluoromethyl)phenyl]methyl}-4-piperidinecarboxamide

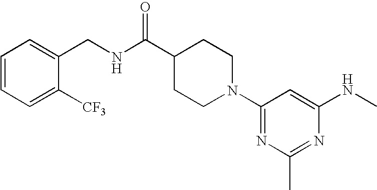

### Step 1: 1-(6-chloro-2-methyl-4-pyrimidinyl)-N-{[2-(trifluoromethyl)phenyl]methyl}-4-piperidinecarboxamide

4,6-dichloro-2-methylpyrimidine (0.3 g, 1.840 mmol) and N-{[2-(trifluoromethyl)phenyl]methyl}-4-piperidinecarboxamide (0.527 g, 1.840 mmol) were combined in a 2.0 mL glass reaction tube that was equipped with a magnetic stir bar. The contents of the tube were taken up in 1,4-dioxane (4 mL), and 1N NaOH (1.840 mL, 1.840 mmol) was added to the room temperature mixture and the contents were allowed to stir vigorously for 60 seconds. The tube was fitted with a rubber septum and hermetically sealed with a crimped metal foil seal. Using a Personal Chemistry Emrys Optimizer microwave unit, the reaction mixture was magnetically stirred and irradiated with microwave energy of dynamically adjusted power in order to maintain a temperature of 180° C. for 1 hour. Once the tube cooled to room temperature the reaction was determined to be complete by LC-MS (m/e 413 [M+1]⁺). Upon standing an off-white solid precipitated out of solution. The reaction mixture was diluted four-fold with water and the solid was collected by vacuum filtration to give 138 mg of 1-(6-chloro-2-methyl-4-pyrimidinyl)-N-{[2-(trifluoromethyl)phenyl]methyl}-4-piperidinecarboxamide (0.334 mmol, 18% yield), was recovered as the free base in the form of an off-white solid. MS (ES) m/e 413 [M+H]+.

### Step 2: 1-[2-methyl-6-(methylamino)-4-pyrimidinyl]-N-{[2-(trifluoromethyl)phenyl]methyl}-4-piperidinecarboxamide

1-(6-chloro-2-methyl-4-pyrimidinyl)-N-{[2-(trifluoromethyl)phenyl]methyl}-4-piperidinecarboxamide (212 mg, 0.514 mmol) and 33 wt. % methylamine in EtOH (4.222 mL, 33.9 mmol) were combined in a 10.0 mL glass reaction tube that was equipped with a magnetic stir bar. The tube was fitted with a rubber septum and hermetically sealed with a crimped metal foil seal. The mixture was stirred at 70° C. for 72 hours. Once the tube cooled to room temperature the reaction was determined to be complete by LC-MS (m/e 408 [M+1]⁺). Upon standing at room temperature the product precipitated out of solution as an off-white solid. The product was isolated by vacuum filtration and the solid was washed with cold EtOH. After drying under vacuum overnight, 78.8 mg of the title compound (0.193 mmol, 38% yield) was recovered in the form of an off-white solid. MS (ES) m/e 408 [M+H]+.

### Example 3

### 1-[6-methyl-2-(methylamino)-4-pyrimidinyl]-N-{[2-(trifluoromethyl)phenyl]methyl}-4-piperidinecarboxamide

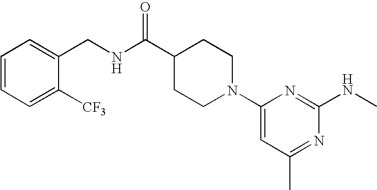

4-chloro-N,6-dimethyl-2-pyrimidinamine (100 mg, 0.370 mmol) and N-{[2-(trifluoromethyl)phenyl]methyl}-4-piperidinecarboxamide (127 mg, 0.443 mmol) were combined in a 5.0 mL glass reaction tube that was equipped with a magnetic stir bar. The contents of the tube were taken up in 1,4-dioxane (1 mL) and 1N NaOH (1.109 mL, 1.109 mmol) was added to the room temperature mixture and the contents were allowed to stir vigorously for 60 seconds. The tube was fitted with a rubber septum and hermetically sealed with a crimped metal foil seal. Using a Personal Chemistry Emrys Optimizer microwave unit, the reaction mixture was magnetically stirred and irradiated with microwave energy of dynamically adjusted power in order to maintain a temperature of 175° C. for 1 hour. Once the tube cooled to room temperature the progress of the reaction was checked by LC-MS which detected the desired product (m/e 408 [M+1]⁺). Upon standing overnight at room temperature a solid precipitated out of solution. The mixture was diluted to 3 times the original volume with water and the product was isolated by vacuum filtration and washed several times with cold H₂O/dioxane. The solid was dried under vacuum at 65° C. for 24 hours to give 122 mg of the title compound (0.296 mmol, 80% yield) as a off-white solid. MS (ES) m/e 408 [M+H]+.

### Example 4

### N-[(2,4-dichlorophenyl)methyl]-1-[6-methyl-2-(methylamino)-4-pyrimidinyl]-4-piperidinecarboxamide

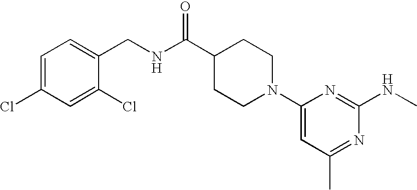

Example 4 was prepared using the general procedure described above in Example substituting N-[(2,4-dichlorophenyl)methyl]-4-piperidine carboxamide for N-{[2-(trifluoromethyl)phenyl]methyl}-4-piperidinecarboxamide. MS (ES) m/e 408 [M+H]+.

As used above, the phrase “using the general procedure described above” indicates that the procedure used employs similar, but not necessarily identical, reaction conditions to those referred to.

The compounds according to Formula I are sEH inhibitors. The compounds according to Formula I, therefore, are useful in the treatment of hypertension and other conditions involving sEH activity. As stated above, mEH provides an important detoxification pathway in mammals. Compounds that exhibit pharmacological selectivity for sEH over mEH therefore are desirable in the methods of treatment described below. Accordingly, in one embodiment the invention is directed to a compound according to Formula I wherein the compound exhibits a selectivity ratio (based on IC50) equal to or greater than 10:1 for sEH over mEH. In another embodiment the invention is directed to a compound according to Formula I wherein the compound exhibits a selectivity ratio (based on IC50) equal to or greater than 100:1 for sEH over mEH. In another embodiment the invention is directed to a compound according to Formula I wherein the compound exhibits a selectivity ratio (based on IC50) equal to or greater than 1000:1 for sEH over mEH.

The biological activity of the compounds according to Formula I can be determined using any suitable assay for determining the activity of a candidate compound as an sEH and/or mEH inhibitor, as well as suitable tissue and/or in vivo models.

### In Vitro Fluorescence Assay

Inhibition of Soluble Expoxide Hydrolase (sEH) activity is measured in a fluorescent assay based upon the format described by Wolf et al. (Analytical Biochemistry Vol. 355 (2006) pp. 71-80). In the presence of sEH, PHOME ((3-Phenyl-oxiranyl)-acetic acid cyano-(6-methoxy-naphthalen-2-yl)-methyl ester), is hydrolyzed to a diol which goes through an intramolecular cyclization and the release and decomposition of cyanohydrin (products=cyanide and 6-methoxy-2-naphthaldehyde). Production of 6-methoxy-2-naphthaldehyde is monitored at excitation of 360 nm and an emission of 465 nm.

The assay is used in a quenched assay format by sequentially adding enzyme (5 uL; 200 pM sEH in 25 mM Hepes at pH 7.0, 0.01% CHAPS (w/v), 0.005% Casein (w/v); 10 minute ambient pre-incubation after addition) then PHOME substrate (5 ul; 10 uM PHOME substrate in 25 mM Hepes at pH 7.0, 0.01% CHAPS (w/v), 0.005% Casein (w/v)) to a 384 well assay plate (Greiner 784076) pre-stamped with 25-100 nL compound at the desired concentration. The reaction is incubated for 30 minutes at room temperature, then quenched by the addition of stop solution (5 uL; 10 mM ZnSO4 in 25 mM Hepes at pH 7.0, 0.01% CHAPS (w/v), 0.005% Casein (w/v)). Microtiter plates are centrifuged after each addition for 30 seconds at 500 rpm. The fluorescence is measured on an EnVision plate reader platform (Perkin Elmer) using a 360 nm excitation filter, 465 nm emission filter, and 400 nm dichroic filter.

Compounds are first prepared in neat DMSO at a concentration of 10 mM, then diluted as required to achieve the desired assay concentration. For inhibition curves, compounds are diluted using a three fold serial dilution and tested at 11 concentrations (e.g. 50 μM-0.8 nM or 25 μM-0.42 nM or 2.5 μM to 42 pM). Curves are analysed using ActivityBase and XLfit, and results are expressed as pIC50 values.

### Cell-Based sEH Inhibitor Assay

Cell based sEH inhibition is measured using the 14,15-DHET immunoassay ELISA kit available from Detroit R&D (Cat. No. DH1), according to the following procedure:
HEK293 cells (BioCat ID 80556) are transduced by sEH BacMam virus to increase sEH expression (other cell lines may be suitable) as follows: One day before the experiment, 1.5 million HEK293 cells (BioCat ID 80556) are seated in 3 ml of DMEM/F12 (with L-Glutamine, with 15 mM HEPES, pH7.30, from Media Prep Lab), with 10% fetal bovine serum (from SAFC Biosciences, Cat. No. 12176-1000M), no antibiotic, in a 25 cm² flask (from Corning Incorporated, Cat. No. 430639) and 30 μL sEH BacMam virus is added. The cells are gently mixed then incubated at 37° C., 5% CO₂, for 24 hours.
The cells are trypsinized to release them from the growth flask, washed once with PBS, then re-suspended in 5 mL DMEM/F12 without phenol red (from Media Prep lab). Cell density should be approximately 3*10⁵ cells/mL (=300 cells/μL), counted using the Cedex AS²⁰ (from Innovatis).
The cells are then diluted in DMEM/F12 to 5.1 cells/□L, and 98 □L/well (=500 cells/well) of this cell suspension is transfered to an assay plate (96 well, clear polystyrene, flat bottom, from Whatman, Cat. No. 7701-1350).
2 □L of the diluted test compound is then added to the cells in the assay plate. The reaction plate is shaken gently and incubated at room temperature for 30 min, after which 10 □L of substrate solution is added (substrate solution is prepared by diluting 1.24□L of 14,15-EET from Cayman Chemical, Cat. No. 50651 with 8.24□L DMEM/F12). The assay plate is then incubated for one hour at room temperature.
After the 1 hour reaction, the reaction mixture is diluted 3 fold with provided sample dilution buffer (ex. Add 220 μL to the 110 μL reaction mixture), mixed well, and spun for 5 min at 500 rpm.
100 μL of the diluted reaction mixture is then transferred from the reaction plates to the ELISA plates, and the ELISA is performed according to the instructions provided in the kit.
IC50s and pIC50s are then calculated. The IC50 can be calculated directly using the 14, 15-DHET concentration or using the % inhibition [% inhibition=100*(1−(sample DHET−0 cell DHET)/(500 cells DHET−0 cell DHET)].
Compounds are first prepared in neat DMSO at a concentration of 0.5 mM, then diluted as required to achieve the desired assay concentration. For inhibition curves, compounds are diluted using a three fold serial dilution and tested at 9 concentrations (e.g. 10 μM-1.5 nM). Curves are analysed using ActivityBase and XLfit, and results are expressed as pIC50 values.

### Biological Activity Results

All exemplified compounds (Examples 1-4) were tested for activity as sEH inhibitors. Where the assay for a particular compound had been performed two or more times, the following conclusion regarding their activities is based on the average of individual experiments: All tested compounds were found to have an IC50 in the range of 0.1 and 10,000 nM.

### Methods of Use

The compounds of the invention inhibit the sEH enzyme and can be useful in the treatment of conditions wherein the underlying pathology is (at least in part) attributable to sEH involvement or in conditions wherein sEH inhibition offers some clinical benefit even though the underlying pathology is not (even in part) attributable to sEH involvement. Examples of such conditions include hypertension, organ failure/damage (including heart failure, renal failure, and liver failure), cardiac and renal fibrosis, peripheral vascular disease (including ischemic limb disease, intermittent claudication, endothelial dysfunction, erectile dysfunction, Raynaud's disease, and diabetic vasculopathies e.g. retinopathy), atherothrombotic disorders (including coronary artery disease, coronary vasospasm, angina, stroke, myocardial ischemia, myocardial infarction, and hyperlipidemia), metabolic disorders (including diabetes), and inflammatory disorders (including arthritis, inflammatory pain, overactive bladder, asthma, and COPD). Accordingly, in another aspect the invention is directed to methods of treating such conditions.

Essential hypertension is commonly associated with the development of significant end organ damage such as renal, endothelial, myocardial, and erectile dysfunction. Such conditions occur “secondary” to the elevated systemic arterial blood pressure. Secondary conditions may be prevented by treatment of the underlying (“primary”) cause. Accordingly, in another aspect the invention is directed to methods of preventing such secondary conditions.

Heart failure is a complex heterogenous disorder characterized by reduced cardiac output, resulting in the inability of the heart to meet perfusion demands of the body. Cardiac proinflammatory cytokine recruitment and maladaptive cardiac hypertrophy, fibrosis and apoptosis/necrosis are factors associated with the progression of heart failure. Compounds of the invention are directed to methods of treating such conditions.

In addition, sEH is indirectly involved in the regulation of platelet function through its effect on EETs. Drugs that inhibit platelet aggregation are believed to decrease the risk of atherthrombotic events, such as myocardial infarction and stroke, in patients with established cardiovascular atherosclerotic disease. Accordingly, in another aspect the invention is directed to methods of preventing atherothrombotic events, such as myocardial infarction and stroke in patients with a history of recent myocardial infarction, stroke, transient ischemic attacks, unstable angina, or atherosclerosis.

The methods of treating and the methods of preventing described above comprise administering a safe and effective amount of a compound of the invention to a patient in need thereof.

As used herein, “treatment” in reference to a condition means: (1) the amelioration or prevention of the condition being treated or one or more of the biological manifestations of the condition being treated, (2) the interference with (a) one or more points in the biological cascade that leads to or is responsible for the condition being treated or (b) one or more of the biological manifestations of the condition being treated, or (3) the alleviation of one or more of the symptoms or effects associated with the condition being treated.

As indicated above, “treatment” of a condition includes prevention of the condition. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof.

As used herein, “safe and effective amount” in reference to a compound of the invention or other pharmaceutically-active agent means an amount of the compound sufficient to significantly induce a positive modification in the condition to be treated but low enough to avoid serious side effects (at a reasonable benefit/risk ratio) within the scope of sound medical judgment. A safe and effective amount of a compound of the invention will vary with the particular compound chosen (e.g. consider the potency, efficacy, and half-life of the compound); the route of administration chosen; the condition being treated; the severity of the condition being treated; the age, size, weight, and physical condition of the patient being treated; the medical history of the patient being treated; the duration of the treatment; the nature of concurrent therapy; the desired therapeutic effect; and like factors, but can nevertheless be determined by the skilled artisan.

As used herein, “patient” refers to a human or other animal.

The compounds of the invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, and subcutaneous injection or infusion. Inhalation refers to administration into the patient's lungs whether inhaled through the mouth or through the nasal passages. Topical administration includes application to the skin as well as intraocular, otic, intravaginal, and intranasal administration.

The compounds of the invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound of the invention depend on the pharmacokinetic properties of that compound, such as absorption, distribution, and half-life, which can be determined by the skilled artisan. In addition, suitable dosing regimens, including the amount administered and the duration such regimens are administered, for a compound of the invention depend on the condition being treated, the severity of the condition being treated, the age and physical condition of the patient being treated, the medical history of the patient to be treated, the nature of concurrent therapy, the particular route of administration chosen, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change. Typical daily dosages range from 1 mg to 1000 mg.

Additionally, the compounds of the invention may be administered as prodrugs. As used herein, a “prodrug” of a compound of the invention is a functional derivative of the compound which, upon administration to a patient, eventually liberates the compound of the invention in vivo. Administration of a compound of the invention as a prodrug may enable the skilled artisan to do one or more of the following: (a) modify the onset of the compound in vivo; (b) modify the duration of action of the compound in vivo; (C) modify the transportation or distribution of the compound in vivo; (d) modify the solubility of the compound in vivo; and (e) overcome or overcome a side effect or other difficulty encountered with the compound. Typical functional derivatives used to prepare prodrugs include modifications of the compound that are chemically or enzymatically cleaved in vivo. Such modifications, which include the preparation of phosphates, amides, esters, thioesters, carbonates, and carbamates, are well known to those skilled in the art.

### Compositions

The compounds of the invention will normally, but not necessarily, be formulated into a pharmaceutical composition prior to administration to a patient. Accordingly, in another aspect the invention is directed to pharmaceutical compositions comprising a compound of the invention and a pharmaceutically-acceptable excipient.

The pharmaceutical compositions of the invention may be prepared and packaged in bulk form wherein a safe and effective amount of a compound of the invention can be extracted and then given to the patient such as with powders, syrups, and solutions for injection. Alternatively, the pharmaceutical compositions of the invention may be prepared and packaged in unit dosage form wherein each physically discrete unit contains a safe and effective amount of a compound of the invention. When prepared in unit dosage form, the pharmaceutical compositions of the invention typically contain from 1 mg to 1000 mg.

The pharmaceutical compositions of the invention typically contain one compound of the invention. However, in certain embodiments, the pharmaceutical compositions of the invention contain more than one compound of the invention. For example, in certain embodiments the pharmaceutical compositions of the invention contain two compounds of the invention. In addition, the pharmaceutical compositions of the invention may optionally further comprise one or more additional pharmaceutically active compounds. Conversely, the pharmaceutical compositions of the invention typically contain more than one pharmaceutically-acceptable excipient. However, in certain embodiments, the pharmaceutical compositions of the invention contain one pharmaceutically-acceptable excipient.

As used herein, “pharmaceutically-acceptable excipient” means a pharmaceutically acceptable material, composition or vehicle involved in giving form or consistency to the pharmaceutical composition. Each excipient must be compatible with the other ingredients of the pharmaceutical composition when comingled such that interactions which would substantially reduce the efficacy of the compound of the invention when administered to a patient and interactions which would result in pharmaceutical compositions that are not pharmaceutically acceptable are avoided. In addition, each excipient must of course be of sufficiently high purity to render it pharmaceutically-acceptable.

The compound of the invention and the pharmaceutically-acceptable excipient or excepients will typically be formulated into a dosage form adapted for administration to the patient by the desired route of administration. For example, dosage forms include those adapted for (1) oral administration such as tablets, capsules, caplets, pills, troches, powders, syrups, elixers, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols and solutions; and (6) topical administration such as creams, ointments, lotions, solutions, pastes, sprays, foams, and gels.

Suitable pharmaceutically-acceptable excipients will vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting the compound or compounds of the invention once administered to the patient from one organ, or portion of the body, to another organ, or portion of the body. Certain pharmaceutically-acceptable excipients may be chosen for their ability to enhance patient compliance.

Suitable pharmaceutically-acceptable excipients include the following types of excipients: Diluents, fillers, binders, disintegrants, lubricants, glidants, granulating agents, coating agents, wetting agents, solvents, co-solvents, suspending agents, emulsifiers, sweetners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically-acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising a safe and effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesuim stearate, calcium stearate, and talc.

1. A compound according to Formula I: 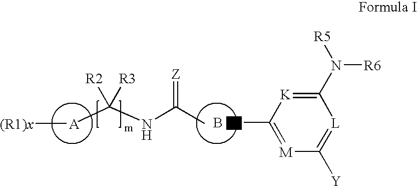
wherein:
A is phenyl, monocyclic heteroaryl, or C5-C6 cycloalkyl;
when A is phenyl or monocyclic heteroaryl each R1 is selected from the group consisting of: halo, —CN, R14, R15, R16, R17, R18, R19, —ORb, —C(O)ORc, —C(O)NRcRc, —NRcRc, —NRcC(O)Rb, —NRcS(O₂)Ra, —SRb, —S(O₂)Ra, and —S(O₂)NRcRc;
when A is C5-C6 cycloalkyl each R1 is selected from the group consisting of: Ra, —ORb, —C(O)ORc, —C(O)NRcRc, —NRcRc, and —NRcC(O)Rb;
each R14 is C1-C6 alkyl optionally substituted with one or more substituents selected from the group consisting of: halo, —ORd, and —NRfRf;
each R15 is C3-C6 cycloalkyl optionally substituted with one or more substituents selected from the group consisting of: halo, —ORd, —NRfRf, and C1-C3 alkyl;
each R16 is monocyclic heterocycloalkyl optionally substituted with one or more C1-C3 alkyl;
each R17 is phenyl optionally substituted with one or more substituents selected from the group consisting of: halo, —CN, C1-C3 alkyl, C1-C3 haloalkyl, —ORd, —NRfR and —S(O₂)Ra;
each R18 is monocyclic heteroaryl optionally substituted with one or more substituents selected from the group consisting of: halo, —CN, C1-C3 alkyl, C1-C3 haloalkyl, —ORd, —NRfR and —S(O₂)Ra;
each R19 is C1-C3 alkyl substituted with R15, R16, R17, or R18;
l is an integer from 0 to 5;
each R2 is H or C1-C3 alkyl;
each R3 is H or C1-C3 alkyl;
m is 1 or 2;
Z is O or S;
B is B1, B2, B3, B4, or B5 wherein
B1 is 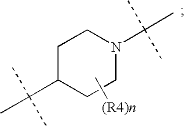
B2 is 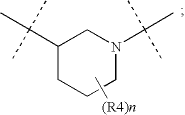
B3 is 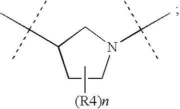
B4 is 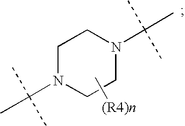
B5 is 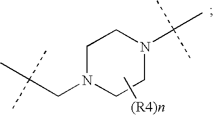
each R4 is C1-C3 alkyl;
n is an integer from 0 to 4;
either K and L are each N and M is CR13, or L and M are each N and K is CR13;
Y is C1-C8 alkyl optionally substituted with one or more substituents selected from the group consisting of: halo, —ORd, —SRd, —NReRe, C3-C6 cycloalkyl, Rh, Ri, and Rj;
R5 is H, R51, R52, R53, R54, R55, —C(O)Rb, —C(O)NRcRc, —S(O₂)Ra, or —S(O₂)NRcRc;
each R51 is C1-C6 alkyl optionally substituted with one or more substituents selected from the group consisting of: halo, —ORd, —SRk, —C(O)ORc, —C(O)NReRe, —NReRe, Rg, Rh, R1, Rj;
R52 is C3-C6 cycloalkyl optionally substituted with one or more substituents selected from the group consisting of: halo, —ORd, —SRd, —C(O)ORc, —C(O)NReRe, —NReRe, C1-C3 alkyl, and C1-C3 haloalkyl;
R53 is monocyclic heterocycloalkyl optionally substituted with one or more C1-C3 alkyl;
R54 is phenyl optionally substituted with one or more substituents selected from the group consisting of: halo, CN, Ra, —ORb, —C(O)ORc, —C(O)NRcRc, —NRcRc, —NRcC(O)Rb, —NRcS(O₂)Ra, —SRb, —S(O₂)Ra, and —S(O₂)NReRe;
R55 is monocyclic heteroaryl optionally substituted with one or more substituents selected from the group consisting of: halo, —CN, C1-C3 alkyl, C1-C3 haloalkyl, —ORd, —NReRe, and —S(O₂)Ra;
R6 is H or R51; or
R5 and R6 taken together with the nitrogen atom to which they are attached form a saturated monocyclic ring having from 5 to 7 member atoms wherein said ring optionally contains one additional heteroatom as a member atom and wherein said ring is optionally substituted with one or more substituents selected from the group consisting of: C1-C3 alkyl, —ORd, and —NRfRf;
R13 is H, R7, R8, R9, R10, R11, —C(O)ORc, —CONRlRl, —NRlRl, —NRcC(O)Rm, —NRcS(O₂)Rm;
R7 is C1-C8 alkyl optionally substituted with one or more substituents selected from the group consisting of: halo, —ORd, —C(O)ORc, —SRd, —NReRe, C3-C6 cycloalkyl, R1, and Rj;
R8 is C3-C6 cycloalkyl optionally substituted with one or more substituents selected from the group consisting of: halo, —ORd, —C(O)ORc, —SRd, —NReRe, C1-C3 alkyl, and C1-C3 haloalkyl;
R9 monocyclic heterocycloalkyl optionally substituted with one or more C1-C3 alkyl;
R10 is phenyl optionally substituted with one or more substituents selected from the group consisting of: halo, CN, Ra, —ORb, —C(O)ORc, —C(O)NReRe, —NReRe, —NRcC(O)Rb, —NRcS(O₂)Ra, —SRb, —S(O₂)Ra, and —S(O₂)NRcRc
R11 is heteroaryl optionally substituted with one or more substituents selected from the group consisting of: halo, CN, Ra, —ORb, —C(O)ORc, —C(O)NReRe, —NReRe, —NRcC(O)Rb, —NRcS(O₂)Ra, —SRb, —S(O₂)Ra, and —S(O₂)NRcRc;
each Ra is C1-C6 alkyl or C1-C6 haloalkyl;
each Rb is H, C1-C6 alkyl or C1-C6 haloalkyl;
each Rc is H or C1-C6 alkyl;
each Rd is H, C1-C3 alkyl or C1-C3 haloalkyl;
each Re is H, C1-C3 alkyl, —CH₂—CF₃; or
both Re groups, independently in each instance, taken together with the nitrogen atom to which they are attached form a saturated monocyclic ring having from 5 to 7 member atoms wherein said ring optionally contains one additional heteroatom as a member atom and wherein said ring is optionally substituted with one or more substituents selected from the group consisting of: C1-C3 alkyl, ORd, and NRfRf;
each Rf is H or C1-C3 alkyl.
each R9 is C3-C6 cycloalkyl optionally substituted with one or more substituents selected from the group consisting of: halo, —ORd, —SRd, —C(O)ORc, —C(O)NReRe, —NReRe, and C1-C3 alkyl;
each Rh is monocyclic heterocycloalkyl optionally substituted with one or more C1-C3 alkyl;
each Ri is phenyl optionally substituted with one or more substituents selected from the group consisting of: halo, —CN, C1-C3 alkyl, C1-C3 haloalkyl, —ORd, —NReRe, and —S(O₂)Ra;
each Rj is monocyclic heteroaryl optionally substituted with one or more substituents selected from the group consisting of: halo, —CN, C1-C3 alkyl, C1-C3 haloalkyl, —ORd, —NReRe, and —S(O₂)Ra;
each Rk is H, C1-C3 alkyl, C1-C3 haloalkyl, or benzyl optionally substituted with one or more substituents selected from the group consisting of: halo, —CN, C1-C3 alkyl, C1-C3 haloalkyl, —ORd, and —NReRe;
each R1 is H, Rh, Ri, Rj, or Rn; or
both R1 groups, independently in each instance, taken together with the nitrogen atom to which they are attached form a saturated monocyclic ring having from 5 to 7 member atoms wherein said ring optionally contains one additional heteroatom as a member atom and wherein said ring is optionally substituted with one or more substituents selected from the group consisting of: C1-C3 alkyl, —ORd, and —NRfRf;
Rm is Rh, R1, Rj, or Rn; and
each Rn is —CH₂—C1-C4 haloalkyl or C1-C6 alkyl optionally substituted with one or more substituents selected from the group consisting of: Rh, R1, and Rj;
or a pharmaceutically acceptable salt thereof.

2. A compound of claim 1 wherein:
A is phenyl, thiophenyl, or pyridyl;
R1 is CF₃, halo, OCF₃, CN, OC₁-C₆ alkyl, morpholino, CO₂H, or N(CH₃)₂;
x is 1, 2, or 3;
B is B1, B2 or B3;
n is 0;
Z is O;
Y is C1-C3 alkyl;
R5 is hydrogen or C1-C6 alkyl;
R6 is hydrogen or C1-C6 alkyl;
K & L are N and M is CR13, or L & M are N and K is CR13;
R13 is hydrogen;
or a pharmaceutically acceptable salt thereof.

3. A compound of claim 1 wherein:
A is phenyl;
R1 is CF₃, halo, OCF₃, CN, OC₁-C₆ alkyl, or morpholino;
x is 1, or 2;
B is B1;
n is 0
Z is O;
Y is methyl;
R5 is hydrogen;
R6 is methyl;
K & L are N and M is CR13, or L & M are N and K is CR13;
R13 is hydrogen;
or a pharmaceutically acceptable salt thereof.

4. A compound of claim 1 chosen from:
N-[(2,4-dichlorophenyl)methyl]-1-[2-methyl-6-(methylamino)-4-pyrimidinyl]-4-piperidinecarboxamide;
1-[2-methyl-6-(methylamino)-4-pyrimidinyl]-N-{[2-(trifluoromethyl)phenyl]methyl}-4-piperidinecarboxamide;
1-[6-methyl-2-(methylamino)-4-pyrimidinyl]-N-{[2-(trifluoromethyl)phenyl]methyl}-4-piperidinecarboxamide; and
N-[(2,4-dichlorophenyl)methyl]-1-[6-methyl-2-(methylamino)-4-pyrimidinyl]-4-piperidinecarboxamide;
or a pharmaceutically acceptable salt thereof.

5. A pharmaceutical composition comprising a compound or salt according to claim 1 and one or more pharmaceutically-acceptable excipient.

6. A method for treating hypertension, heart failure, renal failure, liver failure, peripheral vascular disease, coronary artery disease, myocardial ischemia, angina, or myocardial infarction, comprising administering a safe and effective amount of a compound or salt according to claim 1 to a human in need thereof.

7. A method for preventing stroke comprising administering a safe and effective amount of a compound or salt according to claim 1 to a human in need thereof.

8. A method for treating COPD and asthma comprising administering a safe and effective amount of a compound or salt according to claim 1 to a human in need thereof.

9. A method for treating glucose intolerance, insulin insensitivity, diabetes and obesity comprising administering a safe and effective amount of a compound or salt according to claim 1 to a human in need thereof.
